(19) United States
(12) Patent Application Publication
(10) Pub. No.: US 2004/0019024 A1
(43) Pub. Date: Jan. 29, 2004

(54) Use of vitamin d derivatives as bone resorption inhibitors

(76) Inventors: Seiichi Ishizuka, Tokyo (JP); Kazuya Takenouchi, Tokyo (JP); Atsushi Imaizumi, Tokyo (JP); Yasuhiro Oue, Tokyo (JP); Noriyoshi Kurihara, Pittsburgh, PA (US); Sakamuri V Reddy, Pittsburgh, PA (US); G. David Roodman, Pittsburgh, PA (US)

Correspondence Address: SUGHRUE MION, PLLC, 2100 PENNSYLVANIA AVENUE, N.W., WASHINGTON, DC, 20037, US

(21) Appl. No.: 10/362,565

(22) PCT Filed: Aug. 22, 2001
(86) PCT No.: PCT/US01/22614

(30) Foreign Application Priority Data
Aug. 23, 2000 (JP) 2000-252177

(51) Int. Cl. A61K 31/59

(52) U.S. Cl. 514/167

(57) ABSTRACT

To obtain a bone resorption inhibitor or a treating agent for Paget's disease of bone, there are provided a method of inhibiting bone resorption, comprising administering to a patient a vitamin D antagonist; and a method for treating Paget's disease of bone, comprising administering to a patient a vitamin D antagonist.

### TECHNICAL FIELD

The present invention relates to a group of therapeutic agents containing vitamin D antagonists as their active moiety that inhibits bone resorption. Specifically, the present invention relates to the use of these agents as therapeutic modalities for Paget's disease of bone.

### BACKGROUND ART

Bone is a dynamic tissue that undergoes cycles of resorption followed by new bone formation. This process is continued throughout the skeleton in discreet multi-cellular units called the bone remodeling unit. In the adult bone resorption followed by bone formation is called bone remodeling, and through the remodeling process, bone mass is either maintained or it decreases or increases during life. The relative bone mass depends on the balance between the levels of bone resorption and bone formation. In metabolic bone diseases, this balance between bone resorption and bone formation is changed. Examples include osteoporosis and Paget's disease of bone.

Osteoporosis is a disease in which both the organic components and mineral of bone decrease. In osteoporosis, bone mass decreases, and fractures of bone are more likely to occur. Osteoporosis has been classified into two types; type I, which is, found in postmenopausal women, and type II, which is found in aged persons. In postmenopausal osteoporosis, also called Type I osteoporosis, estrogen deficiency results in increased local levels of cytokines such as interleukin-6, interleukin-11 and interleukin-1. Treatments for postmenopausal osteoporosis include, for example, administration of estrogen or its derivatives, bisphosphonates and calcitonin to block-off osteoclastic bone resorption. Similarly, bone metabolism turnover activators such as 1α-hydroxyvitamin D₃ and 1α,25-hydroxyvitamin D₃ preparations derivatives, and bone formation enhancers such as vitamin K₂ preparations, have all been used to treat this disease.

Paget's disease of bone is the most exaggerated example of abnormal bone remodeling. In Paget's disease of bone, bone resorption is markedly increased, followed by abundant new bone formation. The bone laid down is disorganized in structure and results in weakened bone that can bend and fracture. The primary cellular abnormality in Paget's disease of bone resides in the osteoclast, the bone resorbing cell. In Paget's disease of bone, the osteoclast contains paramyxoviral-like nuclei inclusions, which suggests that Paget's disease of bone may be a slow disease caused by paramyxovirus. In addition, there may be a genetic component to Paget's disease of bone. Up to 40% of these patients have a first degree relative affected with the disease. Paget's disease of bone is the second most common bone disease after osteoporosis and it affects up to 2 to 3 million patients in the United States. Paget's disease of bone is normally treated with bisphosphonate or calcitonin, agents that inhibit osteoclast activity and bone resorption. However, recent findings have suggested there are also abnormalities in 1α,25-dihydroxyvitamin D₃ sensitivity of osteoclast precursors in patients with Paget's disease of bone (J. Bone Miner. Res. vol. 15, 228-236 (2000)). The present inventors have shown that osteoclast precursors from patients with Paget's disease of bone form osteoclasts at concentrations of 1α,25-dihydroxyvitamin D₃ that are 1 to 2 logs less than (10 to 100 times less than) that are required for normal osteoclast formation. Furthermore, these studies have shown that increased sensitivity 1α,25-dihydroxyvitamin D₃ appears to be mediated through induction of a co-activator of the vitamin D receptor. Compounds that interfere with 1α,25-dihydroxyvitamin D₃ binding to its receptor, or the effects of 1α,25-dihydroxyvitamin D₃ on osteoclast precursors, would be a novel and potentially useful agents in treating Paget's disease of bone. If blood levels of 1α,25-dihydroxyvitamin D₃ in Paget's disease of bone patients are similar to normals, these levels of 1α,25-dihydroxyvitamin D₃ may be sufficient to induce bone resorption that would not occur normally. Thus, a pharmaceutical agent that blocks the effects of 1α,25-dihydroxyvitamin D₃ or the hypersensitive osteoclast precursors in Paget's disease of bone, will be very effective therapeutic modalities. Such an agent would have advantages over current therapies for Paget's disease of bone. For example, bisphosphonates in oral form have significant gastrointestinal side effects, and calcitonin therapeutic effectiveness eventually fails over time.

The vitamin D₃ antagonists of the present invention can be synthesized by a method described in the description of international patent publications WO 95/33716 (Compounds of formula (1)), WO 00/24712 (Compounds of formula (1)), WO 94/07853 (Compounds of formula (2)), and WO 97/00242 (Compounds of formula (2)). Compounds of formula (1) directly suppress the effects of 1α,25-dihydroxyvitamin D₃ by inhibiting the binding between 1α,25-dihydroxyvitamin D₃ and 1α,25-dihydroxyvitamin D₃ receptor (VDR) (J. Biol. Chem., vol. 274, 16392-16399 (1999)), the binding between VDR and a the 9-cis-retinoic acid receptor (RXR), and the binding between VDR and a steroid receptor co-activator 1 (SRC-1) of a transcriptional regulation factor (J. Biol. Chem., vol. 274, 32376-32381 (1999). Compounds of formula (2) appear to antagonize the action of 1α,25-dihydroxyvitamin D₃. See and J. Biol. Chem., vol. 275, 16506-16512 (2000)).

### DISCLOSURE OF INVENTION

Inventors of the present invention have found that the vitamin D antagonists strongly suppress osteoclast formation induced by 1α,25-dihydroxyvitamin D₃ by normal bone marrow cells and bone marrow cells from patients with Paget's disease of bone. This decrease in osteoclast formation results in decreased bone resorption. The inventors have also found that vitamin D antagonists suppress bone resorption induced by 1α,25-dihydroxyvitamin D₃ in vitamin D deficient animals, demonstrating that these agents have high suppressive effects on bone resorption both in vitro and in vivo.

It is therefore the object of the present invention to use vitamin D antagonists to suppress bone resorption patients with Paget's disease of bone. These agents should suppress bone resorption without elevating serum calcium levels.

### BEST MODE FOR CARRYING OUT THE INVENTION

Samples of the vitamin D antagonist of the present invention include compounds expressed by the following formula (1),

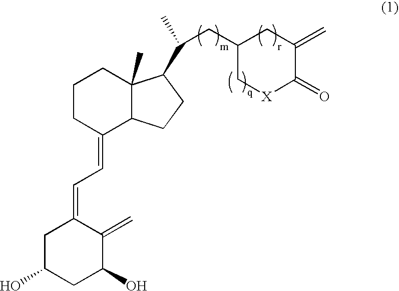

[in formula (1), m is an integer selected from 1 to 3; q is an integer selected from 0 to 3; r is an integer selected from 0 to 3; X is carbon or oxygen; and 1≦q+r≦3]

Among them, a compound whose m is 1 or 2 is preferable. Further, regarding the combinations of m, q, r and X, compounds shown in Table 1 are preferable; and among them, compounds No. 11, 13, 16, 21, 23 and 26 are especially preferable. In the compounds shown in Table 1, if an asymmetric carbon is present in the structure, it includes both the (S) and (R) configurations.

TABLE 1

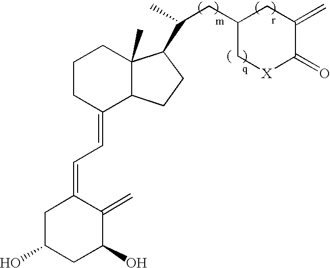

(1)

| Compound No. | m | q | r | X |
|---|---|---|---|---|
| 11 | 1 | 0 | 1 | oxygen |
| 12 | 1 | 1 | 1 | oxygen |
| 13 | 1 | 0 | 1 | carbon |
| 14 | 1 | 0 | 2 | carbon |
| 15 | 1 | 0 | 3 | carbon |
| 16 | 1 | 1 | 0 | carbon |
| 17 | 1 | 2 | 0 | carbon |
| 18 | 1 | 3 | 0 | carbon |
| 21 | 2 | 0 | 1 | oxygen |
| 22 | 2 | 1 | 1 | oxygen |
| 23 | 2 | 0 | 1 | carbon |
| 24 | 2 | 0 | 2 | carbon |
| 25 | 2 | 0 | 3 | carbon |
| 26 | 2 | 1 | 0 | carbon |
| 27 | 2 | 2 | 0 | carbon |
| 28 | 2 | 3 | 0 | carbon |

In addition, samples of the vitamin D antagonist of the present invention include compounds expressed by the following formula (2),

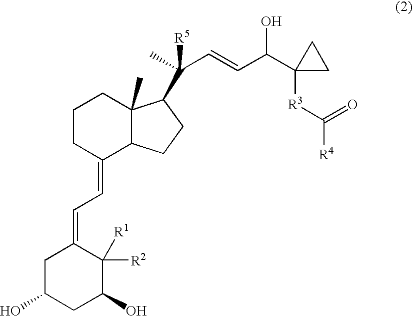

[in formula (2), R¹ and R² are each hydrogen or they together form an exocyclic methylene; R³ is a single bond, methylene or vinylene; R⁴ is a normal or branched C₁ to C₇ alkyl, alkenyl, alkoxy or alkylamino; R⁵ is hydrogen or methyl].

Among them, compounds shown in Table 2 are preferable. In the compounds shown in Table 2, if an asymmetric carbon is present in the structure, the compounds include both the (S) and (R) configurations. When R³ is vinylene, the configuration of the double bond includes both (E)-configuration and (Z)-configuration.

TABLE 2

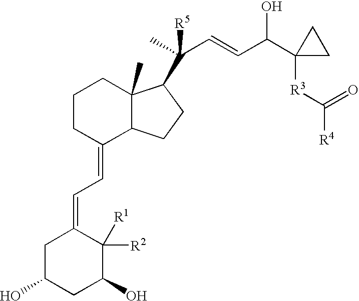

(2)

| Com- | | | | | |
|---|---|---|---|---|---|
| pound | | | | | |
| No. | R¹ | R² | R³ | R⁴ | R⁵ |
| 31 | exocyclic methylene | | single bond | n-butoxy | hydrogen |
| 32 | exocyclic methylene | | single bond | 2-methyl- | hydrogen |
| | | | | propoxy | |
| 41 | exocyclic methylene | | single bond | n-butyl | hydrogen |
| 42 | exocyclic methylene | | single bond | n-pentyl | hydrogen |
| 43 | exocyclic methylene | | single bond | n-heptyl | hydrogen |
| 44 | exocyclic methylene | | single bond | 1-pentenyl | hydrogen |
| 51 | exocyclic methylene | | single bond | n-butylamino | hydrogen |
| 52 | exocyclic methylene | | single bond | n-pentyl- | hydrogen |
| | | | | amino | |
| 53 | exocyclic methylene | | single bond | n-heptyl- | hydrogen |
| | | | | amino | |
| 54 | exocyclic methylene | | single bond | 1-pentenyl- | hydrogen |
| | | | | amino | |
| 61 | exocyclic methylene | | vinylene | ethoxy | hydrogen |
| 62 | exocyclic methylene | | vinylene | t-butoxy | hydrogen |
| 71 | hydrogen | hydrogen | single bond | n-butoxy | hydrogen |
| 81 | hydrogen | hydrogen | vinylene | ethoxy | hydrogen |

It is an object of the present invention to inhibit bone resorption using a vitamin D antagonist without elevating the serum calcium concentration. A second object is to use compositions of the present invention for the treatment of a Paget's disease of bone patient, whose bone resorption activity is extremely accelerated by the action of 1α,25-dihydroxyvitamin D₃.

Inventors of the present invention found that the vitamin D antagonist of the present invention strongly suppresses formation of osteoclasts induced by α,25 dihydroxyvitamin D₃ from bone marrow cells of Paget's disease of bone patients, and suppresses the bone resorption induced by 1α,25-dihydroxyvitamin D₃ in a vitamin D deficient rats.

A bone resorption inhibitor for treating Paget's disease of bone, which contains an above-mentioned compound as the active ingredient, can be formulated as an oral preparation (soft capsules, hard capsules, tablets or a syrup), or in an injectable form with an appropriate vehicle. For example, for treating patients with osteoporosis, an oral preparation may be adequate, but in patients with Paget's disease of bone, who have markedly increased bone resorption, an injectable formulation may be preferable. The injectable formulation should have greater bioavailability.

A vehicle for a parenteral preparation used in the present invention, for example, would be a plant oil, a mineral oil, a white petrolatum, a branched fat or fat-and-oil, a high polymeric alcohol or the like. Among these, a plant oil such as cottonseed oil, corn oil, coconut oil or almond oil is preferable. Oils that contain a medium chain fatty acid as a part of the triglyceride is preferred.

Preferred examples of a vehicle for an oral preparation include cellulose derivatives such as crystalline cellulose, hydroxypropyl cellulose, hydroxypropylmethyl cellulose or methyl cellulose, polyvinyl pyrrolidone, dextrin, cyclodextrin, casein, lactose, mannitol, gelatin or the like.

The amount of an active ingredient for suppressing bone resorption in the present invention is individually decided, depending on the activity of disease, but generally speaking, the amount of an active ingredient is 0.00004 to 0.2 wt. %, preferably 0.0001 to 0.1 wt. %.

The dosage of the active ingredient is also decided depending on the condition of a patient, but generally speaking, it is 0.1 to 1000 μg/day, preferably about 1 to 100 μg/day. The frequency of administration is commonly 1 to 3 times/day. A preparation is preferably formulated in such a manner that these conditions are satisfied.

### EXAMPLES

The present invention will be explained further in detail hereafter with examples; however, it is not restricted by the examples. Further, the Compound No. in each example is the Compound No. shown in the above Table 1.

### Example 1.

Osteoclast Formation-Suppressing Activity of (23S)-25-Dehydro-1α,25-Dihydroxyvitamin D₃-26, 23-Lactone [Compound 11, (23S Isomer)] on the Osteoclast Formation Induced by 1α,25-Dihydroxyvitamin D₃ From Bone Marrows Cells of Normal Persons

Mononuclear cells were fractionated from bone marrow cells of healthy normal persons according to a method of Kurihara et al. (Endocrinology, vol. 126, 2733-2741 (1990)). Briefly, bone marrow cells were obtained from normal persons, a mononuclear cell fraction was collected by Hypaque-Ficoll density gradient centrifugation, the cell fraction was washed with α-Minimal Essential Media (α-MEM) 3 times, and the cells dispersed in α-MEM containing 10% fetal bovine serum. This mononuclear cell suspension was seeded in 100-mm tissue culture plates, the culture plate was kept for 90 minutes at 37° C. in a 4% CO₂-air atmosphere, and the non-adherent cells were collected. The non-adherent mononuclear cells were dispersed into α-MEM culture medium containing 20% horse serum at 10⁶ cells/ml, the cell suspension was seeded into 96-well multi plates at 100 μl/well.

The capacity of the compound 11 (23S isomer) to inhibit osteoclast formation induced by 1α,25-dihydroxyvitamin D₃ was evaluated as follows: Various concentrations of 1α,25-dihydroxyvitamin D₃, various concentrations of compound 11 (23S isomer) or a combination of 10⁻⁸ M 1α,25-dihydroxyvitamin D₃ solution and various concentrations of compound 11 (23S isomer) solution were added to each well. The culture medium was replaced twice weekly and the cultures continued for 3 weeks at 37° C. in an atmosphere of 4% CO₂-air. Osteoclasts that formed were identified by their capacity to bind the 23C6 antibody. The nuclei were counter stained with methyl green. Cells that reacted with the 23C6 antibody and had 3 or more nuclei were scored as osteoclasts. The results are shown in Table 3.

TABLE 3
Compound 11 (23S isomer) inhibit osteoclast formation
induced by 1α.25-dihydroxyvitamin D₃ in normal human bone
marrow cultures.

| | | | osteoclast formation |
|---|---|---|---|
| | | | (average number of |
| compound | concentration | | cells ± S.D.) |
| control (without adding | | | 17 ± 5 |
| test compounds) | | | |
| 1α,25-dihydroxyvitamin D₃ | 10⁻¹¹ | M | 13 ± 3 |
| | 10⁻¹⁰ | M | 11 ± 2 |
| | 10⁻⁹ | M | 34 ± 6 |
| | 10⁻⁸ | M | 87 ± 12 |
| | 10⁻⁷ | M | 91 ± 7 |
| compound 11 (23S isomer) | 10⁻¹¹ | M | 17 ± 4 |
| | 10⁻¹⁰ | M | 9 ± 5 |
| | 10⁻⁹ | M | 5 ± 1 |
| | 10⁻⁸ | M | 3 ± 2 |
| | 10⁻⁷ | M | 0 ± 1 |
| | 10⁻⁶ | M | 0 ± 0 |
| 1α,25-dihydroxyvitamin D₃ | 10⁻⁸ | M | |
| + compound 11 (23S isomer) | 10⁻¹¹ | M | 85 ± 8 |
| + compound 11 (23S isomer) | 10⁻¹⁰ | M | 80 ± 5 |
| + compound 11 (23S isomer) | 10⁻⁹ | M | 55 ± 12 |
| + compound 11 (23S isomer) | 10⁻⁸ | M | 44 ± 8 |
| + compound 11 (23S isomer) | 10⁻⁷ | M | 33 ± 16 |
| + compound 11 (23S isomer) | 10⁻⁶ | M | 20 ± 7 |

1α,25-dihydroxyvitamin D₃ induced osteoclast formation in a dose-dependent manner at 10⁻⁹ M to 10⁻⁷ M concentrations of 1α,25-dihydroxyvitamin D₃. Osteoclast formation was maximal at 10⁻⁸ M 1α,25-dihydroxyvitamin D₃. The compound 11 (23S isomer) did not induce osteoclast formation. When 10⁻⁸ M 1α,25-dihydroxyvitamin D₃ and various concentrations of compound 11 (23S isomer) were added simultaneously to the normal marrow cultures, osteoclast formation induced by the 1α,25-dihydroxyvitamin D₃ was suppressed by compound 11 (23S isomer) at concentration of 10⁻⁹ M to 10⁻⁶ M.

### Example 2.

Effects of (23S)-25-Dehydro-1α,25-Dihydroxyvitamin D₃-26, 23-Lactone [Compound 11, (23S Isomer)] on Osteoclast Formation Induced by 1α,25-Dihydroxyvitamin D₃ in Bone Marrow Cultures From Paget's Disease of Bone Patients.

Bone marrow cells were obtained from involved bones of patients with Paget's disease of bone and processed and cultured as described above for normal bone cells. The cultures were treated in an identical manner with 1α,25-dihydroxyvitamin D₃ and/or compound 11 (23S isomer) as described in Example 1. The results are shown in Table 4.

TABLE 4
Compound 11 (23S isomers inhibit osteoclast formation
induced by 1α.25-dihydroxyvitamin D₃ in bone marrow
cultures from Paaet's disease of bone patients.

| | | | osteoclast formation |
|---|---|---|---|
| | | | (average number of |
| compound | concentration | | cells ± S.D.) |
| control (without adding | | | 47 ± 6 |
| test compounds) | | | |
| 1α,25-dihydroxyvitamin D₃ | 10⁻¹¹ | M | 134 ± 14 |
| | 10⁻¹⁰ | M | 180 ± 15 |
| | 10⁻⁹ | M | 211 ± 25 |
| | 10⁻⁸ | M | 206 ± 16 |
| | 10⁻⁷ | M | 205 ± 6 |
| compound 11 (23S isomer) | 10⁻¹¹ | M | 29 ± 5 |
| | 10⁻¹⁰ | M | 20 ± 4 |
| | 10⁻⁹ | M | 16 ± 2 |
| | 10⁻⁸ | M | 9 ± 3 |
| | 10⁻⁷ | M | 8 ± 2 |
| | 10⁻⁶ | M | 1 ± 1 |
| 1α,25-dihydroxyvitamin D₃ | 10⁻¹⁰ | M | |
| + compound 11 (23S isomer) | 10⁻¹¹ | M | 170 ± 13 |
| + compound 11 (23S isomer) | 10⁻¹⁰ | M | 155 ± 6 |
| + compound 11 (23S isomer) | 10⁻⁹ | M | 125 ± 5 |
| + compound 11 (235 isomer) | 10⁻⁸ | M | 63 ± 5 |
| + compound 11 (235 isomer) | 10⁻⁷ | M | 14 ± 1 |
| + compound 11 (23S isomer) | 10⁻⁶ | M | 1 ± 1 |

In bone marrow cultures from patients with Paget's disease of bone, 1α,25-dihydroxyvitamin D₃ induced osteoclast formation at concentrations as low as 10⁻¹¹ M. 1α,25-dihydroxyvitamin D₃ induced osteoclast formation in these cultures in a dose-dependent fashion from 10⁻¹¹ M to 10⁻⁷ M. Osteoclast formation was maximally in these cultures between 10⁻¹⁰ M and 10⁻⁹ M. These are 1α,25-dihydroxyvitamin D₃, concentrations that are 1 to 2 logs less than that are required for maximal osteoclast formation in normal marrow cultures. Compound 11 (23S isomer) by itself did not induce osteoclast formation and inhibited basal osteoclast formation in the absence of 1α,25-dihydroxyvitamin D₃ or when 1α,25-dihydroxyvitamin D₃ was added. Furthermore, when the 10⁻¹⁰ M 1α,25-dihydroxyvitamin D₃ was added to these cultures in combination with varying concentrations of compound 11 (23S isomer) osteoclast formation was inhibited in dose-dependent fashion between 10⁻¹¹ M and 10⁻⁶ M compound 11 (23S isomer) concentrations. Note that compound 11 (23S isomer) inhibited the increased basal osteoclast formation in cultures of Paget's disease of bone patients, which was not seen in cultures from normal marrow. These data suggest that at physiologic concentrations of 1α,25-dihydroxyvitamin D₃ compound 11 (23S isomer) may return osteoclast formation to normal levels in patients with Paget's disease of bone.

### Example 3.

Osteoclast Inhibitory Capacity of Different Types of Vitamin D Antagonists on Osteoclast Formation Induced by 1α,25-Dihydroxyvitamin D₃ From Bone Marrow Cultures of Paget's Disease of Bone Patients.

Bone marrow cultures were performed as described in Example 1, with the exception that different types of vitamin D antagonists were added to at varying concentrations to bone marrow cultures stimulated with 1α,25-dihydroxyvitamin D₃. The bone marrow cells were taken from involved bones of patients with Paget's disease of bone and the cultures were performed as described in Example 2. There are two diastereoisomers, based on the asymmetric carbon at the 23 position, in compounds 13 and 16. The isomer having the shorter retention time on reverse phase HPLC analysis was the more polar isomer and the isomer having the longer retention time was the less polar isomer. The results are shown in Table 5.

Osteoclast formation-suppressing activities of various
vitamin D antagonists on the osteoclast formation induced
by 1α,25-dihydroxyvitamin D₃ in bone marrow cultures from
Paget's disease of bone patients

| | | | osteoclast |
|---|---|---|---|
| | | | formation (aver- |
| | | | age number of |
| compound | concentration | | cells ± S.D.) |
| control (without adding | | | 47 ± 6 |
| test compounds) | | | |
| 1α, 25-dihydroxyvitamin D₃ | 10⁻¹¹ | M | 134 ± 14 |
| | 10⁻¹⁰ | M | 180 ± 15 |
| | 10⁻⁹ | M | 211 ± 25 |
| | 10⁻⁸ | M | 206 ± 16 |
| compound 11 (23R isomer) | 10⁻⁹ | M | 32 ± 3 |
| | 10⁻⁸ | M | 20 ± 3 |
| | 10⁻⁷ | M | 1 ± 1 |
| compound 13 (more polar isomer) | 10⁻⁹ | M | 44 ± 5 |
| | 10⁻⁸ | M | 25 ± 4 |
| | 10⁻⁷ | M | 3 ± 4 |
| compound 16 (more polar isomer) | 10⁻⁹ | M | 15 ± 2 |
| | 10⁻⁸ | M | 8 ± 3 |
| | 10⁻⁷ | M | 1 ± 1 |
| compound 13 (less polar isomer) | 10⁻⁹ | M | 7 ± 1 |
| | 10⁻⁸ | M | 6 ± 2 |
| | 10⁻⁷ | M | 1 ± 1 |
| 1α,25-dihydroxyvitamin D₃ | 10⁻¹⁰ | M | |
| + compound 11 (23R isomer) | 10⁻⁹ | M | 145 ± 8 |
| + compound 11 (23R isomer) | 10⁻⁸ | M | 70 ± 12 |
| + compound 11 (23R isomer) | 10⁻⁷ | M | 18 ± 9 |
| 1α,25-dihydxoxyvitamin D₃ | 10⁻¹⁰ | M | |
| + compound 13 (more polar isomer) | 10⁻⁹ | M | 152 ± 12 |
| + compound 13 (more polar isomer) | 10⁻⁸ | M | 79 ± 9 |
| + compound 13 (more polar isomer) | 10⁻⁷ | M | 31 ± 5 |
| 1α,25-dihydroxyvitamin D₃ | 10⁻¹⁰ | M | |
| + compound 16 (more polar isomer) | 10⁻⁹ | M | 131 ± 5 |
| + compound 16 (more polar isomer) | 10⁻⁸ | M | 67 ± 7 |
| + compound 16 (more polar isomer) | 10⁻⁷ | M | 18 ± 1 |
| 1α,25-dihydroxyvitamin D₃ | 10⁻¹⁰ | M | |
| + compound 13 (less polar isomer) | 10⁻⁹ | M | 127 ± 4 |
| + compound 13 (less polar isomer) | 10⁻⁸ | M | 70 ± 2 |
| + compound 13 (less polar isomer) | 10⁻⁷ | M | 14 ± 1 |

As shown in Table 5, 1α,25-dihydroxyvitamin D₃ induced osteoclast formation in a dose-dependently manner from 10⁻¹¹ M to 10⁻⁸ M. Compound 11 (23R isomer), compound 13 (more polar isomer and less polar isomer) and compound 16 (more polar isomer) alone did not induce the osteoclast formation, but rather inhibited basal osteoclast formation. Osteoclast formation induced by 10⁻¹⁰ M 1α,25-dihydroxyvitamin D₃ was suppressed by the compound 11 (23R isomer), the compound 13 (more polar isomer or less polar isomer) or compound 16 (more polar isomer) in a dose-dependent manner in the range of 10⁻⁹ M to 10⁻⁷ M. At the concentration of 10⁻⁷ M, osteoclast formation induced by 10⁻¹⁰ M 1α,25-dihydroxyvitamin D₃ was almost completely suppressed by all compounds.

### Example 4.

Measurement of Vitamin D Metabolites in Blood for Paget's Disease of Bone Patients and Normal Persons of a Similar Age

Blood samples (7 to 10 ml) were collected from 9 Paget's disease of bone patients and 10 normal persons of similar ages and the blood samples were held at room temperature for 3 hours. Afterward, they were centrifuged at 3000 rpm for 10 minutes to obtain sera. The concentrations of vitamin D metabolites in the sera were measured by the method of Ishizuka et al. (Journal of Nutritional Science and Vitaminology, vol. 27, 71-75 (1981) and Acta Endocrinology, vol. 104, 96-102 (1983)). Briefly, 3 to 5 ml of each serum sample was diluted 1:3 with water, a chloroform:methanol (1:1) mixed solution in a volume that was two times the volume of the diluted serum was added, and the suspension was vigorously shaken followed by collecting the chloroform layer. The water layer was extracted again with chloroform. The pooled chloroform layers obtained were concentrated on a rotary evaporator, and the residue was placed on a Sephadex LH-20 column (1.2×10 cm) and eluted with a mixed solvent of n-hexane:chloroform:methanol (9:1:1). The 8-17 ml eluent and the 19-60 ml eluent were collected as the 25-OH-D fraction and the 24,25-(OH)₂D+1α,25-(OH)₂D fraction, respectively. Further, the 25-OH-D fraction and the 24,25-(OH)₂D+1α,25-(OH)₂D fraction were each placed on a Zorbax SIL column (4.6×250 mm) and eluted with 12% isopropanol in n-hexane to purify the 25-OH-D, 24,25-(OH)₂D and 1α,25-(OH)₂D fractions. The concentrations of the purified 25-OH-D fraction and 24,25-(OH)₂D fraction were determined by a competitive protein binding assay, using vitamin D binding protein present in serum from a vitamin D deficient rat. The concentration of the purified 1α,25-(OH)₂D was determined by a radioreceptor assay, using the vitamin D receptor from the small intestine of a vitamin D deficient chick. The results are shown in Table 6.

TABLE 6
Measurement of vitamin D metabolites in blood from
Papet's disease of bone patients and normal persons of
similar age

| | serum concentration of vitamin D metabolite | | |
|---|---|---|---|
| | 25-OH-D | 24,25-(OH)₂D | 1α,25-(OH)₂D |
| sample | (ng/ml) | (ng/ml) | (pg/ml) |
| Paget's patients | 40.5 ± 11.1 | 2.64 ± 1.57 | 41.0 ± 9.1 |
| Normals | 39.3 ± 9.5 | 2.39 ± 1.09 | 38.8 ± 12.0 |

In the Table 6, the data are expressed as mean value ±S.D.

The concentrations of vitamin D metabolites in sera from Paget's disease of bone patients did not significantly differ from normals of the similar age. Abnormalities of vitamin D metabolism were not detected in Paget's disease of bone patients. These data show that the concentration of 1α,25-(OH)₂D in sera from Paget's disease of bone patients was 41.0±9.1 pg/ml serum (10⁻¹⁰ M).

As is clear from Examples 1 and 2, osteoclast formation by bone marrow cells from Paget's disease of bone patients, is induced by 10⁻¹⁰ M 1α,25-dihydroxyvitamin D₃. In contrast, osteoclast formation from bone marrow cells of normal persons, is not induced by 10⁻¹⁰ M 1α,25-dihydroxyvitamin D₃. Suda et al. have shown that there is a positive correlation between osteoclast formation and bone resorption (Suda, T and Takahashi, N, Endocrine Review, vol. 20, 345-357 (1999)). That is, compounds stimulating osteoclast formation also affect bone resorption activity.

### Example 5.

Bone Resorption-Suppressing Activity of (23S)-25-Dehydro-1α,25-dihydroxyvitamin D₃-26, 23-Lactone [Compound 11, (23S Isomer] on the Bone Resorption Induced by 1α,25-Dihydroxyvitamin D₃ in Vitamin D Deficient Rats

Four-week-old male Wistar rats purchased from Japan SLC (Shizuoka, in Japan) were used. The animals were kept in wire-net cages (three animals in a cage) and raised for 7 weeks with free access to a vitamin D-deficient diet (Ca, 0.0036%; P, 0.3%; Harlan Taklad Research Diet, Madison, Wis., U.S.A.) and drinking water (well water treated with hypochlorite of 0.4%±0.2 ppm). Temperature was kept at 23±1° C. and the humidity at 55±10%. Five animals were used for each experimental group. The control animals received intravenously administered solvent (5% ethanol-0.1% Triton X-100-physiological saline solution), and the experimental animals were intravenously administered 1α,25-dihydroxyvitamin D₃ at a dose of 0.25 μg/kg. For animals receiving vitamin D derivatives, compound 11 (23S isomer) was administered intravenously at a dose of 2 μg/kg, 10 μg/kg or 50 μg/kg, or the compound 11 (23S isomer) with or without 1α,25-dihydroxyvitamin D₃ at a dose of 0.25 μg/kg. The volume was 2 ml/kg for each animal. 24 hours after administration, a blood sample was taken out from abdominal descending aorta under ether anesthesia, the serum was collected according to conventional methods, and the calcium concentration in the serum was measured. The calcium concentrations were measured according to OCPC-method (Am. J. Clin. Pathol., vol. 45, 290-296 (1966)) with an autoanalyzer (type 7070, manufactured by Hitachi Seisakusho). The results are shown in Table 7.

TABLE 7
Bone resorption-suppressing activity of compound 11 (23S
isomer) on serum calcium levels induced by 1α.25-
dihydroxyvitamin D₃ in vitamin D deficient rats

| | | | serum Ca concentration |
|---|---|---|---|
| compound | dose | | (mg/100 ml serum) |
| control | | | 4.64 ± 0.19 |
| 1α,25-dihydroxyvitamin D₃ | 0.25 | μg/kg | 5.42 ± 0.12*** |
| compound 11 (23S isomer) | 2 | μg/kg | 4.61 ± 0.12 |
| compound 11 (23S isomer) | 10 | μg/kg | 4.67 ± 0.11 |
| compound 11 (23S isomer) | 50 | μg/kg | 4.65 ± 0.18 |
| 1α,25-dihydroxyvitamin D₃ | 0.25 | μg/kg | |
| + compound 11 (23S isomer) | 2 | μg/kg | 5.31 ± 0.22 |
| + compound 11 (23S isomer) | 10 | μg/kg | 4.97 ± 0.18^{a} |
| + compound 11 (23S isomer) | 50 | μg/kg | 4.72 ± 0.21^{b} |

The data are expressed as the mean value±S.D. Significantly different from control: * * * , p<0.001. Significantly different from 1α,25-(OH)₂D₃: a, p<0.01, and b, p<0.01.

The results show that 24 hours after receiving 0.25 μg/kg of 1α,25-dihydroxyvitamin D₃, animals had a significant increased serum calcium concentration compared to controls. Since these rats were raised on a calcium free diet, the increased serum calcium is attributable to the calcium released from bone by osteoclastic bone resorption (Am. J. Physiol., vol. 216, 1351-1359 (1969)). For animals receiving compound 11 (23S isomer), increased serum calcium concentrations were not observed even at a dose of 50 μg/kg. Thus, the compound does not induce bone resorption 24 hours after administration. However, when 1α,25-dihydroxyvitamin D₃ (0.25 μg/kg) and compound 11 (23S isomer) were administered simultaneously at a dose of 2 μg/kg, 10 μg/kg or 50 μg/kg, bone resorption was not increased. These data demonstrate that bone resorption induced 1α,25-dihydroxyvitamin D₃ was suppressed by the administration of compound 11 (23S isomer) in a dose-dependent manner.

These results demonstrate that the vitamin D antagonist of the present invention suppresses bone resorption induced by 1α,25-dihydroxyvitamin D₃ without increasing serum calcium concentrations in vivo. Thus, the vitamin D antagonist of the present invention is useful for treating the increased bone resorption attributable to 1α,25-dihydroxyvitamin D₃ that is seen in Paget's disease of bone patients.

1. A method of inhibiting bone resorption, comprising administering to a patient a vitamin D antagonist.

2. A method for treating Paget's disease of bone, comprising administering to a patient a vitamin D antagonist.

3. The method of claim 1, wherein said antagonist has the formula (1): 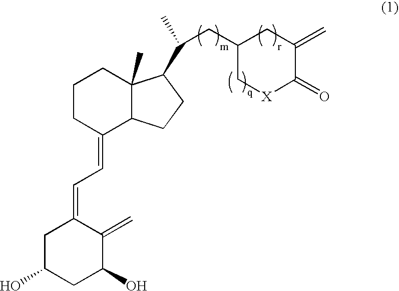
wherein m is an integer selected from 1 to 3; q is an integer selected from 0 to 3; r is an integer selected from 0 to 3; X is carbon or oxygen; and 1≦q+r≦3.

4. The method of claim 2, wherein said antagonist has the formula (1): 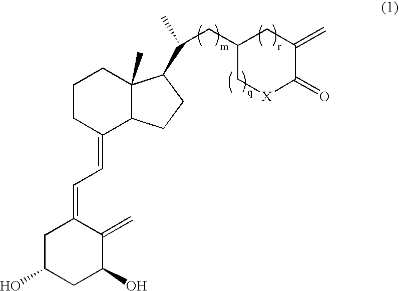
wherein m is an integer selected from 1 to 3; q is an integer selected from 0 to 3; r is an integer selected from 0 to 3; X is carbon or oxygen; and 1≦q+r≦3.

5. The method of claim 3, wherein m is 1 or 2.

6. The method of claim 4, wherein m is 1 or 2.

7. The method of claim 3, wherein m is 1, q is 0, r is 1, and X is oxygen.

8. The method of claim 4, wherein m is 1, q is 0, r is 1, and X is oxygen.

9. The method of claim 3, wherein m is 1, q is 0, r is 1, and X is carbon.

10. The method of claim 4, wherein m is 1, q is 0, r is 1, and X is carbon.

11. The method of claim 3, wherein m is 2, q is 0, r is 1, and X is oxygen.

12. The method of claim 4, wherein m is 2, q is 0, r is 1, and X is oxygen.

13. The method of claim 3, wherein m is 1, q is 1, r is 0, and X is carbon.

14. The method of claim 4, wherein m is 1, q is 1, r is 0, and X is carbon.

15. The method of claim 1, wherein said antagonist has the formula (2): 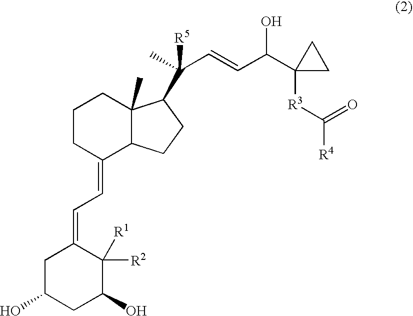
wherein R¹ and R² are each hydrogen or they together form an exocyclic methylene; R³ is a single bond, methylene or vinylene; R⁴ is a normal or branched C₁ to C₇ alkyl, alkenyl, alkoxy or alkylamino; R⁵ is hydrogen or methyl.

16. The method of claim 2, wherein said antagonist has the formula (2): 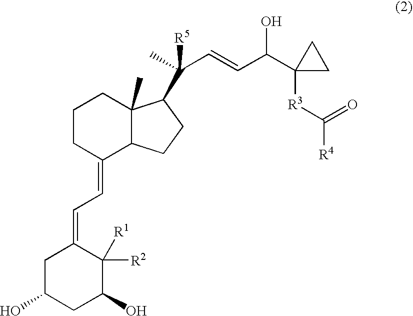
wherein R¹ and R² are each hydrogen or they together form an exocyclic methylene; R³ is a single bond, methylene or vinylene; R⁴ is a normal or branched C₁ to C₇ alkyl, alkenyl, alkoxy or alkylamino; R⁵ is hydrogen or methyl.

17. The method of claim 15, wherein R⁴ is n-butyl, n-pentyl, n-hexyl, n-heptyl, 1-pentenyl, methoxy, ethoxy, n-propoxy, iso-propoxy, 2-methylpropoxy, n-butoxy, t-butoxy, n-pentyloxy, n-hexyloxy or n-heptyloxy, n-butylamino, n-pentylamino, n-heptylamino, n-pentenylamino.

18. The method of claim 16, wherein R⁴ is n-butyl, n-pentyl, n-hexyl, n-heptyl, 1-pentenyl, methoxy, ethoxy, n-propoxy, iso-propoxy, 2-methylpropoxy, n-butoxy, t-butoxy, n-pentyloxy, n-hexyloxy or n-heptyloxy, n-butylamino, n-pentylamino, n-heptylamino, n-pentenylamino.

19. The method of claim 15, wherein R¹ and R² together form an exocyclic methylene; R³ is a single bond; R⁴ is n-butoxy or 2-methylpropoxy; R⁵ is hydrogen.

20. The method of claim 16, wherein R¹ and R² together form an exocyclic methylene; R³ is a single bond; R⁴ is n-butoxy or 2-methylpropoxy; R⁵ is hydrogen.

21. The method of claim 15, wherein R¹ and R² together form an exocyclic methylene; R³ is a single bond; R⁴ is n-butyl, n-pentyl, n-heptyl or 1-pentenyl; R⁵ is hydrogen.

22. The method of claim 16, wherein R¹ and R² together form an exocyclic methylene; R³ is a single bond; R⁴ is n-butyl, n-pentyl, n-heptyl or 1-pentenyl; R⁵ is hydrogen.

23. The method of claim 15, wherein R¹ and R² together form an exocyclic methylene; R³ is a single bond; R⁴ is n-butylamino, n-pentylamino, n-heptylamino or 1-pentenylamino; R⁵ is hydrogen.

24. The method of claim 16, wherein R¹ and R² together form an exocyclic methylene; R³ is a single bond; R⁴ is n-butylamino, n-pentylamino, n-heptylamino or 1-pentenylamino; R⁵ is hydrogen.

25. The method of claim 15, wherein R¹ and R² together form an exocyclic methylene; R³ is vinylene; R⁴ is ethoxy or t-butoxy; R⁵ is hydrogen.

26. The method of claim 16, wherein R¹ and R² together form an exocyclic methylene; R³ is vinylene; R⁴ is ethoxy or t-butoxy; R⁵ is hydrogen.

27. The method of claim 15, wherein R¹ and R² are each hydrogen; R³ is a single bond; R⁴ is n-butoxy; R⁵ is hydrogen.

28. The method of claim 16, wherein R¹ and R² are each hydrogen; R³ is a single bond; R⁴ is n-butoxy; R⁵ is hydrogen.

29. The method of claim 15, wherein R¹ and R² are each hydrogen; R³ is vinylene; R⁴ is ethoxy; R⁵ is hydrogen.

30. The method of claim 16, wherein R¹ and R² are each hydrogen; R³ is vinylene; R⁴ is ethoxy; R⁵ is hydrogen.

31. A pharmaceutical composition for inhibiting bone resorption, comprising a vitamin D antagonist.

32. A pharmaceutical composition for treating Paget's disease of bone, comprising a vitamin D antagonist.

33. The pharmaceutical composition of claim 31, wherein said antagonist has the formula (1): 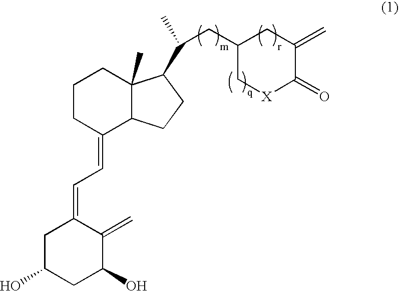
wherein m is an integer selected from 1 to 3; q is an integer selected from 0 to 3; r is an integer selected from 0 to 3; X is carbon or oxygen; and 1≦q+r≦3.

34. The pharmaceutical composition of claim 32, wherein said antagonist has the formula (1): 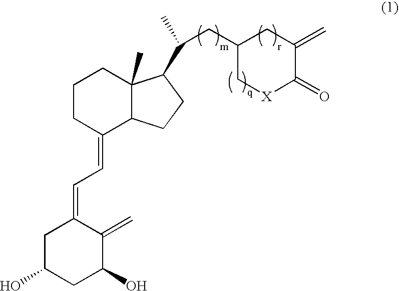
wherein m is an integer selected from 1 to 3; q is an integer selected from 0 to 3; r is an integer selected from 0 to 3; X is carbon or oxygen; and 1≦q+r≦3.

35. The pharmaceutical composition of claim 31, wherein said antagonist has the formula (2): 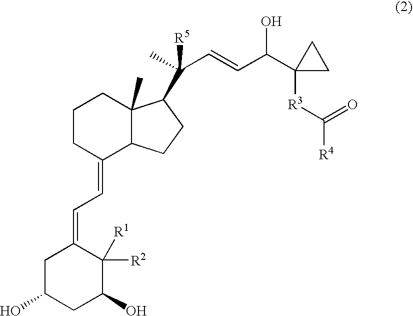
wherein R¹ and R² are each hydrogen or they together form an exocyclic methylene; R³ is a single bond, methylene or vinylene; R⁴ is a normal or branched C₁ to C₇ alkyl, alkenyl, alkoxy or alkylamino; R⁵ is hydrogen or methyl.

36. The pharmaceutical composition of claim 32, wherein said antagonist has the formula (2): 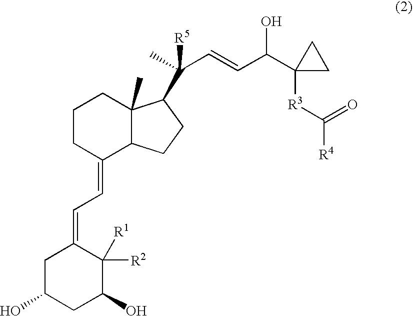
wherein R¹ and R² are each hydrogen or they together form an exocyclic methylene; R³ is a single bond, methylene or vinylene; R⁴ is a normal or branched C₁ to C₇ alkyl, alkenyl, alkoxy or alkylamino; R⁵ is hydrogen or methyl.

37. A use of a vitamin D antagonist for production of a medicament for inhibiting bone resorption.

38. A use of a vitamin D antagonist for production of a medicament for treating Paget's disease of bone.

39. The use of claim 37, wherein said antagonist has the formula (1): 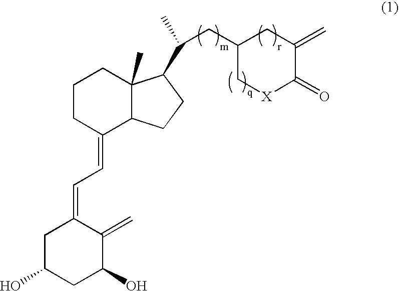
wherein m is an integer selected from 1 to 3; q is an integer selected from 0 to 3; r is an integer selected from 0 to 3; X is carbon or oxygen; and 1≦q+r≦3.

40. The use of claim 38, wherein said antagonist has the formula (1): 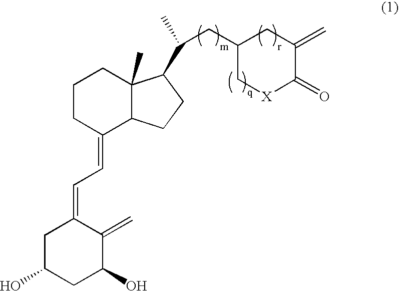
wherein m is an integer selected from 1 to 3; q is an integer selected from 0 to 3; r is an integer selected from 0 to 3; X is carbon or oxygen; and 1≦q+r≦3.

41. The use of claim 37, wherein said antagonist has the formula (2): 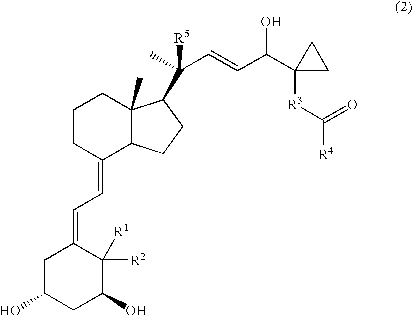
wherein R¹ and R² are each hydrogen or they together form an exocyclic methylene; R³ is a single bond, methylene or vinylene; R⁴ is a normal or branched C₁ to C₇ alkyl, alkenyl, alkoxy or alkylamino; R⁵ is hydrogen or methyl.

42. The use of claim 38, wherein said antagonist has the formula (2): 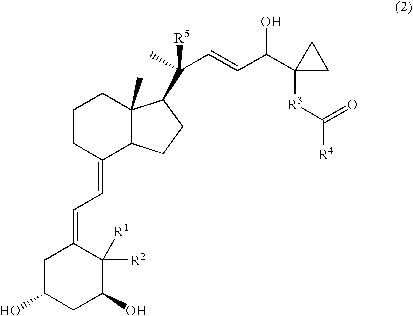
wherein R¹ and R² are each hydrogen or they together form an exocyclic methylene; R³ is a single bond, methylene or vinylene; R⁴ is a normal or branched C₁ to C₇ alkyl, alkenyl, alkoxy or alkylamino; R⁵ is hydrogen or methyl.
